(19) United States
(12) Patent Application Publication
(10) Pub. No.: US 2005/0106076 A1
(43) Pub. Date: May 19, 2005

(54) Ozone-deodorizing toilet

(76) Inventor: Dai-Ming Kuo, Panchiao (TW)

Correspondence Address: Dai-Ming Kuo, P. O. Box 55-846, Taipei, 104, TW

(21) Appl. No.: 10/779,230

(22) Filed: Feb. 11, 2004

(30) Foreign Application Priority Data
Nov. 18, 2003 (TW) 92220348

(51) Int. Cl. A61L 9/015

(52) U.S. Cl. 422/123

(57) ABSTRACT

An ozone-deodorizing toilet includes: an ozone generator provided in a box formed on a rear portion of a toilet seat, a gas outlet formed in a front portion of the box and under the toilet seat, whereby upon seating of a toilet user on the toilet seat and upon actuation of the ozone generator, the ozone as produced from the ozone generator is applied under positive pressure into a bowl chamber as defined among the toilet bowl, the toilet seat and the user's buttocks to allow the ozone to efficiently deodorize and disinfect the stench gas in the bowl chamber of the toilet.

### BACKGROUND OF THE INVENTION

U.S. Pat. No. 6,163,893 disclosed a deodorizing toilet seat pad comprising essentially an ozone-generating means, a toilet seat pad, a base plate, a fan and an active carbon container. When practicing, the fan is operated to draw the stench odor from the toilet into the ozone-generating means where odor is deodorized. Then, the discharged gas is further deodorized by active carbon to remove residual stench.

However, such a deodorizing system may have the following disadvantages:
1. A suction fan should be provided to draw the stench gas to be deodorized by the ozone-generating means and by the active carbon in the active carbon container, requiring complex and costly equipments and increasing operation and maintenance problems.
2. Many perforations (41, 32) are formed in the hollow channel under the toilet seat to be easily accumulated with dirts or bacteria to influence the disinfection effect of the toilet since the bacteria-killing effect is performed in the ozone-generating means located at the gas downstream, not at the upstream such as at the entrance of the perforations (41, 32).
3. Large moisture content in the gas stream as directly sucked from the toilet may decrease the ozone generation efficiency and may quickly saturate the active carbon, thereby minimizing the overall efficiency of toilet deodorization and disinfection.

The present inventor has found the drawbacks of the conventional deodorizing toilet and invented the present ozone-deodorizing toilet.

### SUMMARY OF THE INVENTION

The object of the present invention is to provide an ozone-deodorizing toilet including: an ozone generator provided in a box formed on a rear portion of a toilet seat, a gas outlet formed in a front portion of the box and under the toilet seat, whereby upon seating of a toilet user on the toilet seat and upon actuation of the ozone generator, the ozone as produced from the ozone generator is applied under positive pressure into a bowl chamber as defined among the toilet bowl, the toilet seat and the user's buttocks to allow the ozone to efficiently deodorize and disinfect the stench gas in the bowl chamber of the toilet.

### BRIEF DESCRIPTION OF THE DRAWINGS

FIG. 1 is an illustration showing the application of the present invention.

FIG. 2 is a perspective view of the present invention.

FIG. 3 shows a circuit diagram of the present invention.

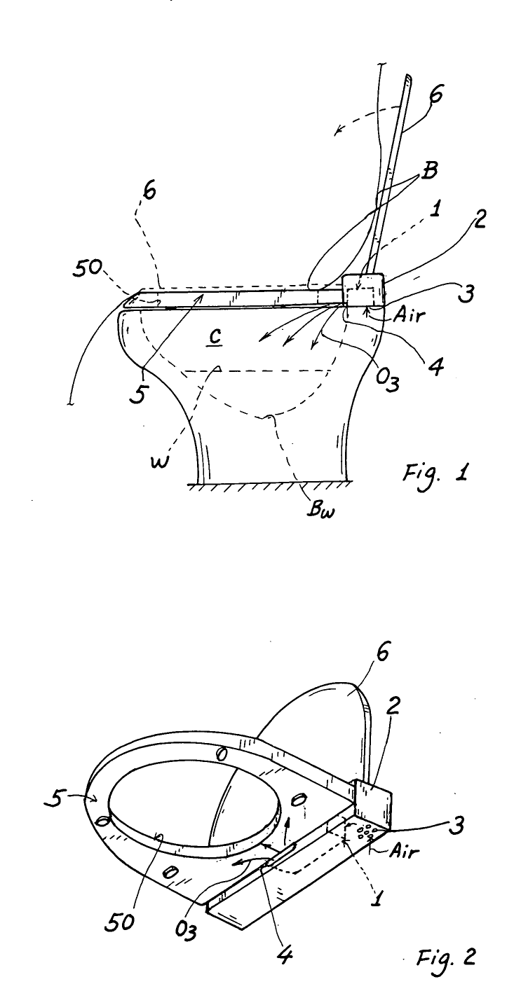

### DETAILED DESCRIPTION

As shown in the drawing figures, the present invention comprises: an ozone generator 1 installed in a box 2 formed on a rear portion of the toilet seat 5, a plurality of air inlet ports 3 formed in a side portion of the box 2 to direct air into the box 2 to be fluidically communicated with the ozone generator 1 for conversion of air into ozone O₃, and a gas outlet port 4 formed in a central front portion of the box 2 and formed under the toilet seat 5 for outwardly downwardly discharging ozone O₃ as produced from the ozone generator 1 into the toilet bowl Bw.

The toilet seat 5 is pivotally formed on a toilet bowl and formed with an opening 50 to be sealed by a toilet user's buttocks B (FIG. 1) or to be closed by a toilet cover 6 as shown in dotted line of FIG. 1.

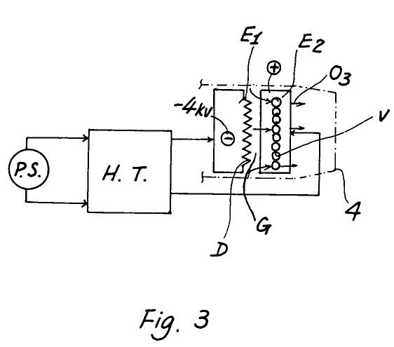

For understanding the operation principles of the ozone generator 1, the circuit diagram as shown in FIG. 3 may be referred.

When the power source P.S. is closed to produce a high tension across the cathode E₁ and anode E₂ through the high-tension generator H.T., the air in the gap G (for example, 3 mn) between the two electrodes E₁, E₂ across the high-tension field will be ionized to form an electronic wind and the air is also partially converted into percentage of ozone O₃, which is then laden in the electronic wind caused by the ionized air and discharged into a bowl chamber C as defined among the toilet bowl Bw, the toilet seat 5, and the user's buttocks B as shown in FIG. 1 to deodorize or disinfect the stench gas in the toilet bowl Bw. The cathode E1 may be −4000 volts, while anode E₂ may be O volt (or other positive volts). A plurality of discharge tips D are juxtapositionally formed on the cathode E₁, each discharge tip D aiming at (or aligned with) each air vent V formed in the anode E₂. The air in the gap G will be ionized to form an electronic wind to flow from E₁ to E₂ and the air is also partially converted into ozone O₃ to be “blown” outwardly though the port 4 driven by the electronic wind (FIG. 3).

Since ozone O₃ is heavier than air, the residual ozone gas, after deodorizing or disinfecting the gas within the bowl chamber C, will be dissolved into water under the water level W to further deodorize or disinfect the waste water in the toilet bowl Bw.

The ozone gas is directed into the bowl chamber C under positive pressure and is, however, confined within the chamber C by the user's buttocks B, the toilet seat 5 and the bowl Bw without serious outward leakage into the environment of a bathroom or rest room.

The air through the gap G in the ozone generator 1 may be converted from an insulator to be a semi-conductor under the high tension field to obtain a percentage of ozone, which may be driven from the first electrode E₁ having a plurality of discharge tips D towards the second electrode E₂ having a plurality of air vents V formed therein (as shown in FIG. 3). The ozone-containing gas may be driven through the holes H and then discharged into the bowl chamber C in the toilet bowl Bw for deodorization and disinfection within the toilet.

The present invention is superior to the conventional deodorizing toilet with the following advantages:
1. The structure is simpler to thereby decrease the cost and maintenance problems.
2. Ozone is directly applied into the toilet bowl to simultaneously deodorize or disinfect the gas and the water in the toilet bowl.
3. The toilet user's buttocks B serves as a “cover” in situ when seating on the toilet to automatically seal the toilet bowl to confine the ozone gas within the bowl for an instant direct deodorization and disinfection within the toilet.
4. The ozone gas is output from the port 4 under positive pressure (by electronic wind of ionized air), to thereby reduce the moisture interference in the ozone generator 1.
5. The ozone gas is blown into the toilet bowl by ionized air (electronic wind) to thereby save or eliminate the air fan, which may increase cost, noise and electromagnetic wave interference.

An automatic electronic system may be provided in the present invention to automatically start the ozone generator in a preset time period so as to emit ozone in a proper time period for satisfactorily deodorization and disinfection in the toilet.

The present invention may be modified without departing from the spirit and scope of the present invention.

1. An ozone-deodorizing toilet comprising:
a toilet seat pivotally formed on a toilet bowl and having a toilet cover pivotally secured to said toilet seat;
a box formed on a rear portion of said toilet seat, having at least an air inlet port formed in said box for directing air into said box, and having a gas outlet port formed in a front portion of said box and positioned under said toilet seat; and
an ozone generator formed in said box and fluidically communicated with said air inlet port, said ozone generator operatively converting the air from said air inlet port to be an ozone gas to be outwardly downwardly discharged, through said outlet port, into said toilet bowl to deodorize and disinfect the stench gas in a bowl chamber defined among said toilet bowl, said toilet seat and a user's buttocks or said toilet cover.

2. A toilet according to claim 1, wherein said ozone generator includes a cathode electrically connected to a negative pole of a power source of high tension having a plurality of discharge tips juxtapositionally formed on said cathode, and an anode electrically connected to a positive pole of said power source and having a plurality of air vents each said air vent aligned with each said discharge tip, defining a gap between each said discharge tip of said cathode and each said air vent of said anode, whereby upon forming of a high voltage across said cathode and said anode to ionize air in said gap to form an electronic wind by ionized air to flow from said cathode to said anode and the air in the gap will be partially converted to ozone, which will be laden in and carried by said electronic wind to be discharged into said toilet bowl for deodorization and disinfection in the toilet.
